(19) United States
(12) Patent Application Publication
(10) Pub. No.: US 2022/0249506 A1
(43) Pub. Date: Aug. 11, 2022

(54) Meloxicam-Containing Granulated Product

(71) Applicant: SSP Co., Ltd., Tokyo (JP)

(72) Inventors: Ryo CHIBA, Chiba (JP); Yoshifumi MURAKI, Tokyo (JP)

(21) Appl. No.: 17/621,960

(22) PCT Filed: Jun. 26, 2020
(86) PCT No.: PCT/JP2020/025252

§ 371 Date: Dec. 22, 2021

(30) Foreign Application Priority Data
Jun. 28, 2019 (JP) 2019-121563

(51) Int. Cl. A61K 31/5415; A61K 9/16

(52) U.S. Cl. CPC A61K 31/5415; A61K 9/1652; A61K 9/1617; A61K 9/1611

(57) ABSTRACT

To provide a technique for improving the dissolution of meloxicam. A granulated product, including the following components (A), (B), and (C): (A) meloxicam or a salt thereof, or a solvate thereof; (B) one or more basic compounds selected from the group consisting of potassium bicarbonate, sodium bicarbonate, arginine, lysine, magnesium hydroxide, and magnesium oxide; and (C) a water-swellable polymer.

### TECHNICAL FIELD

The present invention relates to a granulated product containing meloxicam, and a solid preparation containing the granulated product.

### BACKGROUND ART

Many of nonsteroidal anti-inflammatory drugs (NSAIDs) suppress the formation of inflammation-related prostaglandins, thromboxanes, and prostacyclins by inhibiting cyclooxygenase (COX), as an anti-inflammatory action mechanism. In the COX, there are two kinds of COXs of COX1 which is constantly produced in most of the organs such as stomach and kidney, and COX2 which is specifically produced at an inflamed site. The NSAIDs have COX1-inhibiting ability and often faces a problem that a gastrointestinal disorder in the stomach or the like easily occurs as the side effect.

Meanwhile, meloxicam is a selective COX2 inhibitor, and therefore considered to be unlikely to cause a gastrointestinal disorder. Therefore, there are many use cases of meloxicam as a therapeutic agent for pain in patients suffering from inflammatory disease.

However, meloxicam is almost insoluble in water in a low pH range of pH 5.0 or less, and is difficult to dissolve when the meloxicam is made into a solid preparation, and thus a problem has been that the bioavailability of the solid preparation is low, and the pain relief starts slowly.

Patent Literature 1 discloses granules in which dihydroxy aluminum aminoacetate (aluminum glycinate), magnesium carbonate, and the like are added to meloxicam, and a capsule obtained by filling a capsule with granules in which magnesium aluminometasilicate and the like are added to meloxicam, but there has been still room for improvement in the dissolution of meloxicam.

### CITATION LIST

### Patent Literature

[PTL 1] Japanese unexamined patent application publication no 2012-21025

### SUMMARY OF INVENTION

### Technical Problem

An object of the present invention is to provide a technique for improving the dissolution of meloxicam.

### Means for Solving the Problem

In view of this, as a result of intensive studies to solve the problems described above, the present inventors have found that the dissolution of meloxicam is improved by granulating one or more basic compounds selected from the group consisting of potassium bicarbonate, sodium bicarbonate, arginine, lysine, magnesium hydroxide, and magnesium oxide, and a water-swellable polymer in combination, in addition to meloxicam or a salt thereof, or a solvate thereof; and thus completed the present invention.

That is, the present invention is to provide the following <1> to <7>.

<1> A granulated product, comprising the following components (A), (B), and (C):

(A) meloxicam or a salt thereof, or a solvate thereof;

(B) one or more basic compounds selected from the group consisting of potassium bicarbonate, sodium bicarbonate, arginine, lysine, magnesium hydroxide, and magnesium oxide; and

(C) a water-swellable polymer.

<2> The granulated product according to <1>, wherein the component (B) is one or more basic compounds selected from the group consisting of potassium bicarbonate, sodium bicarbonate, arginine, lysine, and magnesium oxide.

<3> The granulated product according to <1> or <2>, wherein a content mass ratio of the component (B) to the component (A), [(B)/(A)], is 0.1 or more and 250 or less.

<4> The granulated product according to any one of <1> to <3>, wherein the component (C) is one or more water-swellable polymers selected from the group consisting of low-substituted hydroxypropylcellulose, carmellose, a salt of carmellose, croscarmellose, a salt of croscarmellose, carboxymethyl starch, a salt of carboxymethyl starch, and crystalline cellulose.

<5> The granulated product according to any one of <1> to <4>, wherein a content mass ratio of the component (C) to the component (A), [(C)/(A)], is 0.1 or more and 50 or less.

<6> A solid preparation, comprising the granulated product according to any one of <1> to <5>.

<7> A method for improving dissolution of meloxicam or a salt thereof, or a solvate thereof, comprising granulating a composition comprising the following components (A), (B), and (C):

(A) meloxicam or a salt thereof, or a solvate thereof;

(B) one or more basic compounds selected from the group consisting of potassium bicarbonate, sodium bicarbonate, arginine, lysine, magnesium hydroxide, and magnesium oxide; and

(C) a water-swellable polymer.

### Effects of the Invention

According to the present invention, the dissolution of meloxicam can be improved.

### DETAILED DESCRIPTION OF THE INVENTION

<Granulated Product>

A granulated product according to the present invention comprises the following components (A), (B), and (C):

(A) meloxicam or a salt thereof, or a solvate thereof;

(B) one or more basic compounds selected from the group consisting of potassium bicarbonate, sodium bicarbonate, arginine, lysine, magnesium hydroxide, and magnesium oxide; and

(C) a water-swellable polymer.

(Component (A))

Examples of the meloxicam or a salt thereof, or a solvate thereof include meloxicam; an alkali metal salt of meloxicam, such as a sodium salt, and a potassium salt; an ammonium salt of meloxicam; a meglumine salt of meloxicam; a tris salt of meloxicam; and a salt of meloxicam with a basic amino acid: and a hydrate or an alcoholate thereof.

The meloxicam or a salt thereof, or a solvate thereof, can be produced by a known method, and a commercially available product can also be used.

From the viewpoint of the dissolution of meloxicam, the content of the meloxicam or a salt thereof, or a solvate thereof is preferably 0.5 to 50% by mass, more preferably 1 to 25% by mass, furthermore preferably 1 to 15% by mass, still more preferably 1.5 to 12.5% by mass, and particularly preferably 2 to 7.5% by mass, with respect to the total mass of the granulated product according to the present invention.

(Component (B))

From the viewpoint of the dissolution of meloxicam, the basic compound of the component (B) is preferably one or more selected from the group consisting of potassium bicarbonate, sodium bicarbonate, arginine, lysine, and magnesium oxide.

In this regard, the arginine and lysine as the component (B) may include salts of arginine and lysine as well as free forms of arginine and lysine. Examples of the salts of arginine and lysine include inorganic acid salts such as a hydrochloride, a sulfate, and a nitrate; and organic acid salts such as an acetate, and a tartrate. Specific examples of the arginine and lysine include L-arginine, L-arginine hydrochloride, L-lysine, L-lysine hydrochloride, and L-lysine acetate.

The basic compound as the component (B) may be used singly, or in combinations of two or more thereof.

The basic compound as the component (B) can be produced by a known method, and a commercially available product can also be used.

From the viewpoint of the dissolution of meloxicam, the content of the basic compound as the component (B) is preferably 0.5 to 80% by mass, more preferably 1 to 60% by mass, furthermore preferably 3 to 50% by mass, still more preferably 5 to 40% by mass, and particularly preferably 10 to 30% by mass, with respect to the total mass of the granulated product according to the present invention.

In addition, from the viewpoint of the dissolution of meloxicam or the like, the content mass ratio of the component (B) to the component (A), [(B)/(A)], is preferably 0.1 or more, more preferably 0.5 or more, furthermore preferably 0.75 or more, still more preferably 1 or more, still furthermore preferably 1.5 or more, and particularly preferably 3 or more. Further, from the viewpoint of the dissolution of meloxicam or the like, the content mass ratio [(B)/(A)] is preferably 250 or less, more preferably 100 or less, furthermore preferably 50 or less, still more preferably 20 or less, still furthermore preferably 15 or less, even more preferably 10 or less, and particularly preferably 7.5 or less.

(Component (C))

In the present invention, the expression “water-swellable polymer” refers to a polymer poorly soluble in water, which swells with the addition of water or hydrous alcohol and can swell by retaining a large amount of water or hydrous alcohol.

Examples of the water-swellable polymer include low-substituted hydroxypropylcellulose, carmellose, a salt of carmellose, croscarmellose, a salt of croscarmellose, carboxymethyl starch, a salt of carboxymethyl starch, and crystalline cellulose. These may be used singly, or in combinations of two or more thereof.

Examples of the salt of carmellose include an alkali metal salt or alkaline earth metal salt of carmellose, such as carmellose sodium, and carmellose calcium. Examples of the salt of croscarmellose include an alkali metal salt or alkaline earth metal salt of croscarmellose, such as croscarmellose sodium, and croscarmellose calcium. Examples of the salt of carboxymethyl starch include an alkali metal salt of carboxymethyl starch, such as sodium carboxymethyl starch.

In the low-substituted hydroxypropylcellulose, from the viewpoint of the dissolution of meloxicam, the content of the hydroxypropoxy group is preferably 5 to 16% by mass, the content of the hydroxypropoxy group is more preferably 6 to 13% by mass, and the content of the hydroxypropoxy group is particularly preferably 7 to 10% by mass.

Examples of the commercially available product of the low-substituted hydroxypropylcellulose include L-HPC (LH31) manufactured by Shin-Etsu Chemical Co., Ltd. (hydroxypropoxy group: 11% by mass), L-HPC (LH11) manufactured by ShinEtsu Chemical Co., Ltd. (hydroxypropoxy group: 11% by mass), L-HPC (LH32) manufactured by Shin-Etsu Chemical Co., Ltd. (hydroxypropoxy group: 8% by mass), and L-HPC (NBD-020) manufactured by Shin-Etsu Chemical Co., Ltd. (hydroxypropoxy group: 14% by mass). Further, the average particle diameter of the low-substituted hydroxypropylcellulose is usually 60 μm or less, and preferably 45 μm or less and particularly preferably 4 to 25 μcm from the viewpoint of the dissolution of meloxicam.

Among the water-swellable polymers as described above, from the viewpoint of the dissolution of meloxicam, low-substituted hydroxypropylcellulose, carmellose, a salt of carmellose, croscarmellose, a salt of croscarmellose, carboxymethyl starch, and a salt of carboxymethyl starch are preferred, low-substituted hydroxypropylcellulose, carmellose, a salt of carmellose, croscarmellose, and a salt of croscarmellose are more preferred, a salt of carmellose is furthermore preferred, and carmellose calcium is particularly preferred.

From the viewpoint of the dissolution of meloxicam, the content of the water-swellable polymer is preferably 1 to 95% by mass, more preferably 10 to 90% by mass, furthermore preferably 20 to 80% by mass, still more preferably 30 to 75% by mass, and particularly preferably 40 to 70% by mass with respect to the total mass of the granulated product according to the present invention.

In addition, from the viewpoint of the dissolution of meloxicam or the like, the content mass ratio of the component (C) to the component (A), [(C)/(A)], in the granulated product according to the present invention is preferably 0.1 or more, more preferably 0.75 or more, furthermore preferably 1 or more, still more preferably 2 or more, and particularly preferably 5 or more. Further, from the viewpoint of the dissolution of meloxicam or the like, the content mass ratio [(C)/(A)] is preferably 50 or less, more preferably 25 or less, furthermore preferably 20 or less, still more preferably 17.5 or less, and particularly preferably 15 or less.

In addition, from the viewpoint of the dissolution of meloxicam or the like, the content mass ratio of the component (C) to the component (B), [(C)/(B)] in the granulated product according to the present invention is preferably 0.05 or more, more preferably 0.15 or more, furthermore preferably 1 or more, and particularly preferably 2 or more. Further, from the viewpoint of the dissolution of meloxicam or the like, the content mass ratio [(C)/(B)] is preferably 50 or less, more preferably 25 or less, furthermore preferably 15 or less, still more preferably 10 or less, and particularly preferably 5 or less.

The granulated product according to the present invention can be roughly classified into granules, fine granules, and powders depending on the particle size. The average particle diameter of the granulated product according to the present invention is preferably 50 to 1,000 μcm, and more preferably 50 to 500 μcm. The average particle diameter can be measured by a sieving method.

<Solid Preparation>

The solid preparation according to the present invention contains the granulated product according to the present invention as described above.

The contents of the components (A) to (C) with respect to the total mass of the solid preparation according to the present invention are not particularly limited, and are preferably in the ranges similar to those of the contents of the components (A) to (C) with respect to the total mass of the granulated product according to the present invention. Further, the content mass ratios [(B)/(A)], [(C)/(A)], and [(C)/(B)] in the solid preparation according to the present invention are also preferably in the ranges similar to those of the content mass ratios [(B)/(A)], [(C)/(A)], and [(C)/(B)] in the granulated product according to the present invention, respectively.

The solid preparation according to the present invention may be a solid preparation obtained by using the granulated product according to the present invention as a raw material (intermediate product). Examples of the dosage form of the solid preparation according to the present invention include granules, fine granules, powders, tablets (uncoated tablets, orally disintegrating (OD) tablets, chewable tablets, dispersible tablets, soluble tablets, troches, sublingual tablets, buccal tablets, mucoadhesive tablets, effervescent tablets, medicated chewing gums, etc.), pills, capsules (soft capsules, hard capsules, etc.), dry syrups, and jellies. Further, these may be coated with sugar, film or the like in accordance with a known method. The average particle diameter of granules is preferably 50 to 1,000 μcm, and more preferably 50 to 500 μcm. The average particle diameter can be measured by a sieving method.

In this regard, the solid preparation according to the present invention is preferably a solid preparation for oral use.

Examples of the specific form of the solid preparation according to the present invention include the following (α-1) to (α-3).

(α-1) A solid preparation, which is obtained by making the granulated product according to the present invention directly into granules, fine granules, powders, or the like, and subjecting them to coating processing as needed.

(α-2) A capsule, which is obtained by filling a capsule with the granulated product according to the present invention.

(α-3) A tablet, which is obtained by tableting the granulated product according to the present invention through a compression method or the like.

The granulated product and solid preparation according to the present invention may contain a drug other than the above-described components depending on the intended purpose. Examples of the drug include an antacid (excluding the component (B)), an anti-inflammatory agent (excluding the component (A)), a sedative drug, caffeines, an antitussive expectorant, an antihistamine, an antiallergic agent, an anticholinergic drug, vitamins, a muscle relaxant, and herbal medicines. These may be used singly, or in combinations of two or more thereof.

Examples of the antacid include calcium carbonate, precipitated calcium carbonate, calcium silicate, synthetic aluminum silicate, magnesium carbonate, dibasic calcium phosphate, anhydrous dibasic calcium phosphate, aluminum hydroxide gel, dried aluminum hydroxide gel, aluminum hydroxide-magnesium carbonate mixed dried gel, an aluminum hydroxide-magnesium carbonate-calcium carbonate coprecipitation product, glycine, magnesium silicate, synthetic hydrotalcite, magnesium aluminometasilicate, and aluminum glycinate. These may be used singly, or in combinations of two or more thereof.

The content of the antacid is preferably 0 to 5% by mass, and more preferably 0 to 0.01% by mass, with respect to the total mass of the granulated product or solid preparation according to the present invention.

Examples of the anti-inflammatory agent include glycyrrhizic acid or a salt thereof, tranexamic acid, glycyrrhetinic acid, sodium azulene sulfonate, aspirin, and salicylamide. These may be used singly, or in combinations of two or more thereof.

Examples of the sedative drug include allylisopropylacetylurea, and bromovalerylurea. As the sedative drug, these may be used singly, or in combinations of two or more thereof.

The content of the sedative drug is preferably 0 to 10% by mass, more preferably 0 to 1% by mass, and particularly preferably 0 to 0.01% by mass, with respect to the total mass of the granulated product or solid preparation according to the present invention.

Examples of the caffeines include caffeine hydrate, anhydrous caffeine, and caffeine and sodium benzoate. These may be used singly, or in combinations of two or more thereof.

The content of the caffeines is preferably 0 to 5% by mass, and more preferably 0 to 1% by mass, with respect to the total mass of the granulated product or solid preparation according to the present invention.

Further, the granulated product and solid preparation according to the present invention may contain pharmaceutical additives other than the above-described components. Examples of the pharmaceutical additives include an excipient such as lactose, starch, white soft sugar, glucose, mannitol, sorbitol, and xylitol; a binder such as polyvinyl pyrrolidone, hydroxypropyl methyl cellulose, polyvinyl alcohol, a polyvinyl alcohol-acrylic acid-methyl methacrylate copolymer, and pullulan; a lubricant such as magnesium stearate, talc, calcium stearate, stearyl fumarate, and a sucrose fatty acid ester; a fluidizer such as light anhydrous silicic acid, hydrous silicon dioxide, corn starch, and magnesium aluminometasilicate; and a disintegrant such as pregelatinized starch, and crospovidone. These may be used singly, or in combinations of two or more thereof. Further, a dissolution aid, a buffer, a preservative, a flavor, a dyestuff, a flavoring agent, and the like can be used, as needed.

The content of the pharmaceutical additives is preferably 0 to 70% by mass, and more preferably 0 to 50% by mass, with respect to the total mass of the granulated product or solid preparation according to the present invention.
In this regard, in the granulated product and solid preparation according to the present invention, the water content is preferably 0 to 5% by mass, and more preferably 0 to 2.5% by mass.

The dose of the granulated product or solid preparation according to the present invention is preferably a dose at which 1 to 30 mg of meloxicam can be taken per day, more preferably a dose at which 2.5 to 15 mg of meloxicam can be taken per day, and particularly preferably a dose at which 5 to 10 mg of meloxicam can be taken per day. Further, the number of doses is preferably once to three times per day, and more preferably once per day.

<Method for Producing Granulated Product, and Method for Improving Dissolution>

The granulated product according to the present invention can be produced by a method involving a granulation step of granulating a composition containing components (A), (B), and (C) (hereinafter, also referred to as “composition X”). According to this method and a method for producing a solid preparation using this method, a granulated product and a solid preparation, in each of which the dissolution of meloxicam or a salt thereof, or a solvate thereof is improved, can be easily produced.

In this regard, in the present specification, the expression “improving dissolution” refers to that meloxicam or a salt thereof, or a solvate thereof becomes easy to dissolve from a solid preparation.

The content mass ratios [(B)/(A)], [(C)/(A)], and [(C)/(B)] in a composition X are preferably in the ranges similar to the content mass ratios [(B)/(A)], [(C)/(A)], and [(C)/(B)] in the granulated product according to the present invention, respectively. In this regard, the order of mixing the components (A) to (C) into the composition X does not matter.

In addition, the composition X can be prepared by, for example, a technique of mixing components (A) to (C) and other components (the above-described drugs and pharmaceutical additives) as needed, or a technique of mixing components (A) to (C) and other components (the above-described drugs and pharmaceutical additives) as needed and then kneading the obtained mixture and a solvent such as water or hydrous alcohol together. As the hydrous alcohol, a hydrous alcohol having an alcohol content of 30% by mass or less is preferred. Further, as the alcohol, a lower alcohol such as ethanol, and 2-propanol is preferred. The amount of the above solvent to be used is preferably 0.5 to 3 times by mass with respect to the total amount of the components (A) to (C). In addition, the above-described mixing and kneading can be performed by using, for example, a stirring-type mixer or the like. It is preferred to perform each of the mixing and the kneading at 20 to 1,200 rpm for 0.5 to 10 minutes.

It is preferred to perform the above granulation by a wet granulation method. Examples of the wet granulation method include a stirring granulation method, a fluidized bed granulation method, and an extrusion granulation method, and an extrusion granulation is preferred.

In addition, drying treatment, pulverization treatment, sieving, spheroidizing treatment by a Marumerizer, coating treatment with sugars, polymers, etc., and the like may be appropriately selected and performed on the granulated product obtained as described above. The granulated product can be used directly as granules, fine granules, or powders. Further, by tableting the granulated product in accordance with a conventional method, a tablet can be obtained, and by filling a capsule with the granulated product in accordance with a conventional method, a capsule can be obtained.

In addition, the granulated product according to the present invention and a solid preparation containing the granulated product, each have extremely high dissolution of meloxicam or a salt thereof, or a solvate thereof, even in a low pH range (for example, pH 1 to 5), and therefore, it can be expected that the anti-inflammatory action, the analgesic action, and the antipyretic action are instantaneously obtained when such a granulated product or a solid preparation is orally administered. In particular, prompt anti-inflammation and analgesia for rheumatoid arthritis, osteoarthritis, low back pain, shoulder periarthritis, or cervico-omo-brachial syndrome can be expected.

### EXAMPLES

Hereinafter, the present invention will be described in detail with reference to Examples, however, the present invention is not limited to the following Examples.

### Reference Example 1: Granules

Meloxicam (manufactured by Combi-Blocks Inc.), corn starch (manufactured by Matsutani Chemical Industry Co., Ltd.), and low-substituted hydroxypropylcellulose (L-HPC (LH31), manufactured by Shin-Etsu Chemical Co., Ltd.) each were weighed so as to have the mixing amounts shown in the following Table 1 per sample of granules and so as to be 40 g in total. These were charged into a granulator (MM-20N manufactured by OKADASEIKO Co., Ltd.), and mixed at 900 rpm for 3 minutes. After that, an adequate amount of purified water was added into a granulator (MM-20N manufactured by OKADASEIKO Co., Ltd.), the kneading was performed at 900 rpm for 3 minutes, and then the extrusion granulation (0.8 mmp screen) was performed to obtain a granular material. The obtained granular material was dried by a box-type dryer (operated at 65° C. overnight). By sizing the dried granular material, granules of Reference Example 1 (16 to 60 mesh) were prepared.

### Examples 1 to 6: Granules

Granules (16 to 60 mesh) were prepared in a similar manner as in Reference Example 1 except that the corn starch was changed to sodium bicarbonate, potassium bicarbonate, magnesium hydroxide, magnesium oxide, arginine, and lysine, respectively.

### Comparative Examples 1 to 9: Granules

Granules (16 to 60 mesh) were prepared in a similar manner as in Reference Example 1 except that the corn starch was changed to sodium citrate, calcium silicate, calcium carbonate, magnesium carbonate, synthetic hydrotalcite (manufactured by Tomita Pharmaceutical Co., Ltd.), dried aluminum hydroxide gel (manufactured by Tomita Pharmaceutical Co., Ltd.), magnesium aluminometasilicate (Neusilin A FP, manufactured by Fuji Chemical Industries Co., Ltd.), aluminum glycinate (GLYCINAL, manufactured by Kyowa Chemical Industry Co., Ltd.), and glycine, respectively.

### Reference Example 2: Mixture

Meloxicam (manufactured by Combi-Blocks Inc.), corn starch (manufactured by Matsutani Chemical Industry Co., Ltd.), and low-substituted hydroxypropylcellulose (L-HPC (LH31), manufactured by Shin-Etsu Chemical Co., Ltd.) each were weighed so as to have the mixing amounts shown in the following Table 2 per sample of mixture and so as to be 40 g in total. These were charged into a granulator (MM-20N manufactured by OKADASEIKO Co., Ltd.), and mixed at 900 rpm for 3 minutes, and thus a mixture of Reference Example 2 was prepared.

### Comparative Examples 10 to 18: Mixture

A mixture was prepared in a similar manner as in Reference Example 2 except that the corn starch was changed to sodium bicarbonate, potassium bicarbonate, magnesium hydroxide, magnesium oxide, arginine, lysine, dried aluminum hydroxide gel (manufactured by Tomita Pharmaceutical Co., Ltd.), aluminum glycinate (GLYCINAL, manufactured by Kyowa Chemical Industry Co., Ltd.), and glycine, respectively.

TABLE 1

| | Reference | | | | | | | | | | | | | | | |
|---|---|---|---|---|---|---|---|---|---|---|---|---|---|---|---|---|
| | Example | Example | | | | | | Comparative Example | | | | | | | | |
| Granules (mg) | 1 | 1 | 2 | 3 | 4 | 5 | 6 | 1 | 2 | 3 | 4 | 5 | 6 | 7 | 8 | 9 |
| Meloxicam | 10 | 10 | 10 | 10 | 10 | 10 | 10 | 10 | 10 | 10 | 10 | 10 | 10 | 10 | 10 | 10 |
| Sodium bicarbonate | | 50 | | | | | | | | | | | | | | |
| Potassium bicarbonate | | | 50 | | | | | | | | | | | | | |
| Magnesium hydroxide | | | | 50 | | | | | | | | | | | | |
| Magnesium oxide | | | | | 50 | | | | | | | | | | | |
| Arginine | | | | | | 50 | | | | | | | | | | |
| Lysine | | | | | | | 50 | | | | | | | | | |
| Sodium citrate | | | | | | | | 50 | | | | | | | | |
| Calcium silicate | | | | | | | | | 50 | | | | | | | |
| Calcium carbonate | | | | | | | | | | 50 | | | | | | |
| Magnesium carbonate | | | | | | | | | | | 50 | | | | | |
| Synthetic hydrotalcite | | | | | | | | | | | | 50 | | | | |
| Dried aluminum hydroxide | | | | | | | | | | | | | 50 | | | |
| gel | | | | | | | | | | | | | | | | |
| Magnesium | | | | | | | | | | | | | | 50 | | |
| aluminometasilicate | | | | | | | | | | | | | | | | |
| Aluminum glycinate | | | | | | | | | | | | | | | 50 | |
| Glycine | | | | | | | | | | | | | | | | 50 |
| Low-substituted | 120 | 120 | 120 | 120 | 120 | 120 | 120 | 120 | 120 | 120 | 120 | 120 | 120 | 120 | 120 | 120 |
| hydroxypropylcellulose | | | | | | | | | | | | | | | | |
| (LH-31) | | | | | | | | | | | | | | | | |
| Corn starch | 50 | | | | | | | | | | | | | | | |
| Total | 180 | 180 | 180 | 180 | 180 | 180 | 180 | 180 | 180 | 180 | 180 | 180 | 180 | 180 | 180 | 180 |

TABLE 2

| | Reference | | | | | | | | | |
|---|---|---|---|---|---|---|---|---|---|---|
| | Example | Comparative Example | | | | | | | | |
| Mixture (mg) | 2 | 10 | 11 | 12 | 13 | 14 | 15 | 16 | 17 | 18 |
| Meloxicam | 10 | 10 | 10 | 10 | 10 | 10 | 10 | 10 | 10 | 10 |
| Sodium bicarbonate | | 50 | | | | | | | | |
| Potassium bicarbonate | | | 50 | | | | | | | |
| Magnesium hydroxide | | | | 50 | | | | | | |
| Magnesium oxide | | | | | 50 | | | | | |
| Arginine | | | | | | 50 | | | | |
| Lysine | | | | | | | 50 | | | |
| Dried aluminum hydroxide gel | | | | | | | | 50 | | |
| Aluminum glycinate | | | | | | | | | 50 | |
| Glycine | | | | | | | | | | 50 |
| Low-substituted hydroxypropylcellulose | 120 | 120 | 120 | 120 | 120 | 120 | 120 | 120 | 120 | 120 |
| (LH-31) | | | | | | | | | | |
| Corn starch | 50 | | | | | | | | | |
| Total | 180 | 180 | 180 | 180 | 180 | 180 | 180 | 180 | 180 | 180 |

### Test Example 1

McIlvaine buffer was diluted with purified water to obtain 900 mL of liquid having pH 4.0. Into the liquid, 180 mg of granules of Reference Example 1 was added, and the obtained mixture was stirred at 50 rpm at 37° C. After the lapse of 5 minutes, 10 minutes, and 30 minutes from the start of stirring, the dissolution rate of meloxicam in the granules of Reference Example 1 was measured.

The dissolution rate of meloxicam was measured by a high performance liquid chromatography (HPLC) method, and calculated from the dissolution amount of meloxicam in accordance with the following equation (a).

Meloxicam dissolution rate (% by mass)={(meloxicam dissolution amount (mg))/(meloxicam initial content (mg))}×100 (α)

### <HPLC Measurement Conditions>

Column: stainless steel tube having an inner diameter of 4.6 mm and a length of 15 cm, filled with 5 μm of octadecylsilylated silica gel for liquid chromatography

Detector: ultraviolet absorptiometer (measurement wavelength: 353 nm)

Mobile phase: diluted phosphoric acid (1→1,000)/acetonitrile mixture (1:1)

Further, also for the granules or mixture of each of Examples 1 to 6, Comparative Examples 1 to 18, and Reference Example 2, the dissolution rate of meloxicam was measured in a similar manner as in the above.

The results are shown in Table 3. In this regard, it can be deemed that the larger the dissolution rate after the lapse of 30 minutes is, the better the dissolution of meloxicam is.

TABLE 3

| | | | Dissolution rate (% by mass) | | |
|---|---|---|---|---|---|
| | | | After 5 | After 10 | After 30 |
| | | Components | minutes | minutes | minutes |
| Reference Example 1 | Granules | Corn starch | 0.0 | 0.0 | 1.1 |
| Example 1 | Granules | Sodium bicarbonate | 72.6 | 74.1 | 75.6 |
| Example 2 | Granules | Potassium bicarbonate | 81.2 | 83.2 | 81.6 |
| Example 3 | Granules | Magnesium hydroxide | 13.2 | 20.7 | 34.9 |
| Example 4 | Granules | Magnesium oxide | 14.6 | 26.6 | 51.6 |
| Example 5 | Granules | Arginine | 49.3 | 62.1 | 65.6 |
| Example 6 | Granules | Lysine | 71.8 | 77.3 | 80.2 |
| Comparative Example 1 | Granules | Sodium citrate | 5.9 | 6.2 | 6.9 |
| Comparative Example 2 | Granules | Calcium silicate | 2.3 | 4.7 | 11.1 |
| Comparative Example 3 | Granules | Calcium carbonate | 2.2 | 3.2 | 4.9 |
| Comparative Example 4 | Granules | Magnesium carbonate | 12.0 | 16.0 | 20.0 |
| Comparative Example 5 | Granules | Synthetic hydrotalcite | 9.3 | 12.5 | 15.6 |
| Comparative Example 6 | Granules | Dried aluminum hydroxide gel | 1.0 | 1.4 | 1.8 |
| Comparative Example 7 | Granules | Magnesium aluminometasilicate | 2.5 | 4.2 | 6.0 |
| Comparative Example 8 | Granules | Aluminum glycinate | 1.3 | 2.2 | 3.3 |
| Comparative Example 9 | Granules | Glycine | 0.5 | 1.0 | 3.0 |
| Reference Example 2 | Mixture | Corn starch | 0.7 | 1.5 | 3.7 |
| Comparative Example 10 | Mixture | Sodium bicarbonate | 3.9 | 4.7 | 6.4 |
| Comparative Example 11 | Mixture | Potassium bicarbonate | 1.0 | 1.6 | 4.0 |
| Comparative Example 12 | Mixture | Magnesium hydroxide | 4.3 | 6.1 | 10.0 |
| Comparative Example 13 | Mixture | Magnesium oxide | 10.8 | 13.6 | 14.4 |
| Comparative Example 14 | Mixture | Arginine | 5.7 | 7.2 | 8.9 |
| Comparative Example 15 | Mixture | Lysine | 10.9 | 12.2 | 12.9 |
| Comparative Example 16 | Mixture | Dried aluminum hydroxide gel | 0.8 | 1.5 | 3.9 |
| Comparative Example 17 | Mixture | Aluminum glycinate | 0.7 | 1.7 | 4.0 |
| Comparative Example 18 | Mixture | Glycine | 0.8 | 1.6 | 4.0 |

### Example 7: Granules

Granules (16 to 60 mesh) were prepared in a similar manner as in Example 1 except that the mixing amount of the low-substituted hydroxypropylcellulose was set so as to be 7.5 mg.

### Examples 8 to 9: Granules

Granules (16 to 60 mesh) were prepared in a similar manner as in Example 1 except that the mixing amount of the sodium bicarbonate was set so as to be 20 mg, and 100 mg, respectively.

### Test Example 2

For the granules of each of Examples 7 to 9, the dissolution rate of meloxicam was measured in a similar manner as in Test Example 1.

The results are shown in Table 4.

TABLE 4

| | | Example 1 | Example 7 | Example 8 | Example 9 |
|---|---|---|---|---|---|
| Meloxicam | | 10 mg | 10 mg | 10 mg | 10 mg |
| Sodium bicarbonate | | 50 mg | 50 mg | 20 mg | 100 mg |
| Low-substituted | | 120 mg | 7.5 mg | 120 mg | 120 mg |
| hydroxypropylcellulose | | | | | |
| (LH31) | | | | | |
| Total | | 180 mg | 67.5 mg | 150 mg | 230 mg |
| Dissolution rate | After 5 | 72.6 | 39.4 | 35.7 | 40.6 |
| (% by mass) | minutes | | | | |
| | After 10 | 74.1 | 49.9 | 39.2 | 43.0 |
| | minutes | | | | |
| | After 30 | 75.6 | 50.8 | 41.2 | 42.7 |
| | minutes | | | | |

### Examples 10 to 16: Granules

Granules (16 to 60 mesh) were prepared in a similar manner as in Example 1 except that the L-HPC (LH31) manufactured by Shin-Etsu Chemical Co., Ltd. was changed to L-HPC (LH11) manufactured by Shin-Etsu Chemical Co., Ltd. (low-substituted hydroxypropylcellulose), L-HPC (LH32) manufactured by Shin-Etsu Chemical Co., Ltd. (low-substituted hydroxypropylcellulose), NBD-020 manufactured by Shin-Etsu Chemical Co., Ltd. (low-substituted hydroxypropylcellulose), croscarmellose sodium (Ac-Di-Sol, manufactured by FMC International), carmellose (NS-300, manufactured by Nichirin Chemical Industries, Ltd.), carmellose calcium (E.C.G-505, manufactured by Nichirin Chemical Industries, Ltd.), and sodium carboxymethyl starch (Primojel, manufactured by DFE pharma), respectively.

### Comparative Example 19: Granules

Granules (16 to 60 mesh) were prepared in a similar manner as in Example 1 except that the L-HPC (LH31) manufactured by Shin-Etsu Chemical Co., Ltd. was changed to lactose hydrate (200M, manufactured by DFE pharma).

### Test Example 3

For the granules of each of Examples 10 to 16 and Comparative Example 19, the dissolution rate of meloxicam was measured in a similar manner as in Test Example 1.

The results are shown in Table 5.

TABLE 5

| | | Example | Example | Example | Example | Example | Example | Example | Example | Comparative |
|---|---|---|---|---|---|---|---|---|---|---|
| | | 1 | 10 | 11 | 12 | 13 | 14 | 15 | 16 | Example 19 |
| Meloxicam | | 10 mg | 10 mg | 10 mg | 10 mg | 10 mg | 10 mg | 10 mg | 10 mg | 10 mg |
| Sodium bicarbonate | | 50 mg | 50 mg | 50 mg | 50 mg | 50 mg | 50 mg | 50 mg | 50 mg | 50 mg |
| Low-substituted | | 120 mg | | | | | | | | |
| hydroxypropylccllulose (LH31) | | | | | | | | | | |
| Low-substituted | | | 120 mg | | | | | | | |
| hydroxypropylcellulose (LH11) | | | | | | | | | | |
| Low-substituted | | | | 120 mg | | | | | | |
| hydroxypropylcellulose (LH32) | | | | | | | | | | |
| Low-substituted | | | | | 120 mg | | | | | |
| hydroxypropylcellulose (NBD-020) | | | | | | | | | | |
| Croscarmellose sodium | | | | | | 120 mg | | | | |
| Carmellose | | | | | | | 120 mg | | | |
| Carmellose calcium | | | | | | | | 120 mg | | |
| Sodium carboxymethyl starch | | | | | | | | | 120 mg | |
| Lactose hydrate | | | | | | | | | | 120 mg |
| Total | | 180 mg | 180 mg | 180 mg | 180 mg | 180 mg | 180 mg | 180 mg | 180 mg | 180 mg |
| Dissolution rate | After 5 | 72.6 | 68.5 | 83.0 | 52.8 | 54.9 | 72.6 | 60.9 | 56.2 | 23.9 |
| (% by mass) | minutes | | | | | | | | | |
| | After 10 | 74.1 | 70.9 | 85.4 | 60.1 | 64.2 | 72.3 | 97.9 | 55.2 | 24.1 |
| | minutes | | | | | | | | | |
| | After 30 | 75.6 | 73.8 | 84.2 | 62.5 | 68.8 | 68.1 | 98.3 | 46.8 | 23.0 |
| | minutes | | | | | | | | | |

### Production Examples 1 to 6: Granules

Respective components weighed in accordance with each of the granule prescriptions shown in Table 6 were charged into a high-speed stirring granulator (Model: VG-10, manufactured by Powrex Corporation), and mixed for 3 minutes to obtain a mixed powder. Subsequently, purified water was added to the mixed powder, and the obtained mixture was kneaded for 3 minutes with a high-speed stirring granulator (Model: VG-10, manufactured by Powrex Corporation). The obtained kneaded material was granulated by using an extrusion granulator (TDG-80 type, manufactured by DALTON CORPORATION). Further, the obtained extruded granules were charged into a fluidized-bed dryer (Model: FLO-2, manufactured by Freund Corporation), dried until the water content of the granules was 2% or less, and then the sizing was performed by a size reduction machine (Model: QC-U10, manufactured by Powrex Corporation) to obtain the granules of each of Production Examples 1 to 6.

TABLE 6

| | Granule prescription (g) | | | | | |
|---|---|---|---|---|---|---|
| | Production | Production | Production | Production | Production | Production |
| Component name | Example 1 | Example 2 | Example 3 | Example 4 | Example 5 | Example 6 |
| Meloxicam | 60 | 60 | 60 | 60 | 60 | 60 |
| Potassium bicarbonate | 300 | | | | | |
| Sodium bicarbonate | | 300 | | | | |
| L-arginine | | | 300 | | | |
| L-lysine | | | | 300 | | |
| Magnesium hydroxide | | | | | 300 | |
| Magnesium oxide | | | | | | 300 |
| Low-substituted | 720 | 720 | 720 | 720 | 720 | 720 |
| hydroxypropylcellulose | | | | | | |
| Total | 1080 | 1080 | 1080 | 1080 | 1080 | 1080 |

### Production Examples 7 to 12: Film-Coated Tablet

To 900 g of the granules obtained in each of Production Examples 1 to 6, 140 g of crystalline cellulose, and 10 g of magnesium stearate were added, and these materials were mixed using a V-type mixer (TCV-5 type, manufactured by TOKUJU CORPORATION) to obtain 1,050 g of tableting mixture. Next, the tableting mixture was tableted using a rotary tableting machine (Model: VIRGO 512, manufactured by Kikusui Seisakusho Ltd.) to produce uncoated tablets each having a diameter of 7.5 mm and a tablet mass of 210 mg (meloxicam content: 10 mg).

Subsequently, 120 g of hypromellose and 20 g of macrogol 6000 were dissolved in purified water, and then into the obtained mixture, 10 g of talc and 10 g of titanium oxide were dispersed to prepare 2,000 g of film-coating liquid having a solid content concentration of 8%. Next, 500 g of uncoated tablets were charged into a coating machine (Model: HC-LABO, manufactured by Freund Corporation), and sprayed with the above-prepared film-coating liquid to perform the coating, and thus film-coated tablets of each of Production Examples 7 to 12 (each having a tablet mass of 220 mg and white color) were obtained.

### Production Examples 13 to 18: Hard Capsule

To 900 g of the granules obtained in each of Production Examples 1 to 6, 9 g of magnesium stearate was added, and these materials were mixed using a V-type mixer (TCV-5 type, manufactured by TOKUJU CORPORATION) to obtain 909 g of granule mixture for filling. Next, No. 4 gelatin capsules were filled with the obtained granule mixture by using a capsule filling machine (Model: LIQUFIL-5, manufactured by Qualicaps Co., Ltd.) to obtain hard capsules of each of Production Examples 13 to 18 (each having a capsule content of 181.8 mg, corresponding to 10 mg of meloxicam content).

1. A granulated product, comprising following components (A), (B), and (C):
(A) meloxicam or a salt thereof, or a solvate thereof;
(B) one or more basic compounds selected from the group consisting of potassium bicarbonate, sodium bicarbonate, arginine, lysine, magnesium hydroxide, and magnesium oxide; and
(C) a water-swellable polymer.

2. The granulated product according to claim 1, wherein
the component (B) is one or more basic compounds selected from the group consisting of potassium bicarbonate, sodium bicarbonate, arginine, lysine, and magnesium oxide.

3. The granulated product according to claim 1, wherein
a content mass ratio of the component (B) to the component (A), [(B)/(A)], is 0.1 or more and 250 or less.

4. The granulated product according to claim 1, wherein
the component (C) is one or more water-swellable polymers selected from the group consisting of low-substituted hydroxypropylcellulose, carmellose, a salt of carmellose, croscarmellose, a salt of croscarmellose, carboxymethyl starch, a salt of carboxymethyl starch, and crystalline cellulose.

5. The granulated product according to claim 1, wherein
a content mass ratio of the component (C) to the component (A), [(C)/(A)], is 0.1 or more and 50 or less.

6. A solid preparation, comprising the granulated product according to claim 1.

7. A method for improving dissolution of meloxicam or a salt thereof, or a solvate thereof, comprising granulating a composition comprising following components (A), (B), and (C):
(A) meloxicam or a salt thereof, or a solvate thereof;
(B) one or more basic compounds selected from the group consisting of potassium bicarbonate, sodium bicarbonate, arginine, lysine, magnesium hydroxide, and magnesium oxide; and
(C) a water-swellable polymer.

8. The granulated product according to claim 2, wherein a content mass ratio of the component (B) to the component (A), [(B)/(A)], is 0.1 or more and 250 or less.

9. The granulated product according to claim 8, wherein the component (C) is one or more water-swellable polymers selected from the group consisting of low-substituted hydroxypropylcellulose, carmellose, a salt of carmellose, croscarmellose, a salt of croscarmellose, carboxymethyl starch, a salt of carboxymethyl starch, and crystalline cellulose.

10. The granulated product according to claim 3, wherein the component (C) is one or more water-swellable polymers selected from the group consisting of low-substituted hydroxypropylcellulose, carmellose, a salt of carmellose, croscarmellose, a salt of croscarmellose, carboxymethyl starch, a salt of carboxymethyl starch, and crystalline cellulose.

11. The granulated product according to claim 2, wherein a content mass ratio of the component (C) to the component (A), [(C)/(A)], is 0.1 or more and 50 or less.

12. The granulated product according to claim 3, wherein a content mass ratio of the component (C) to the component (A), [(C)/(A)], is 0.1 or more and 50 or less.

13. The granulated product according to claim 4, wherein a content mass ratio of the component (C) to the component (A), [(C)/(A)], is 0.1 or more and 50 or less.

14. A solid preparation, comprising the granulated product according to claim 2.

15. A solid preparation, comprising the granulated product according to claim 3.

16. A solid preparation, comprising the granulated product according to claim 4.

17. A solid preparation, comprising the granulated product according to claim 5.

18. The method of claim 7, wherein the component (B) is one or more basic compounds selected from the group consisting of potassium bicarbonate, sodium bicarbonate, arginine, lysine, magnesium hydroxide, and magnesium oxide.

19. The method of claim 7, wherein the component (C) is one or more water-swellable polymers selected from the group consisting of low-substituted hydroxypropylcellulose, carmellose, a salt of carmellose, croscarmellose, a salt of croscarmellose, carboxymethyl starch, a salt of carboxymethyl starch, and crystalline cellulose.
